(19) United States
(12) Patent Application Publication
(10) Pub. No.: US 2020/0375935 A1
(43) Pub. Date: Dec. 3, 2020

(54) ENHANCED DELIVERY OF A HYDROPHOBIC AGENT IN THE ORAL CAVITY BY COUPLING TO A HYDROPHILIC AGENT WITH CELLULAR ACTIVITY

(71) Applicant: Gary R. Jernberg, Mankato, MN (US)

(72) Inventor: Gary R. Jernberg, Mankato, MN (US)

(21) Appl. No.: 16/868,437

(22) Filed: May 6, 2020

Related U.S. Application Data
(60) Provisional application No. 62/854,052, filed on May 29, 2019.
(60) Provisional application No. 62/879,034, filed on Jul. 26, 2019.

(51) Int. Cl. A61K 31/201; A61K 47/12; A61K 45/06; A61K 47/36; A61K 9/00

(52) U.S. Cl. CPC A61K 31/201; A61K 47/12; A61K 9/006; A61K 47/36; A61K 45/06

(57) ABSTRACT

The compounds herein may be used to form an oral treatment composition. An oral treatment composition according to the present disclosure may comprise a mixture of different ingredients that when combined together result in an improved delivery mechanism to treat teeth and soft tissues of oral cavity. Generally the invention can be used to treat an oral disorder by delivering an oral treatment composition to a surface within the oral cavity, wherein the oral treatment composition comprises a hydrophobic active agent and a hydrophilic agent. The hydrophobic active agent may be a bacterial biofilm dispersion agent, a bacterial biofilm inhibitor, or a fatty acid signaling agent.

### CROSS REFERENCE TO RELATED APPLICATIONS

This application claims the benefit of U.S. Application No. 62/854,052, filed May 29, 2019 and U.S. Application Nos. 62/854,052 62/879,034, filed Jul. 26, 2019, the disclosures of which are hereby incorporated by reference in their entirety.

### INTRODUCTION

Dental biofilm, or dental plaque, is a diverse community of microorganisms found on the tooth surface and gingiva embedded in an extracellular matrix of host and microbial polymers. Dental plaque can occur above (supragingival) and below (subgingival) the gumline. Plaques that form on subgingival tooth surfaces and coat the epithelium lining of the gingival crevice lead to the development of periodontal infections (i.e., gingivitis and periodontitis). Bacteria in dental biofilm are the major cause of several oral diseases including gingivitis, chronic and aggressive periodontitis, and necrotizing periodontal diseases. For example, bacterium Porphyromonas gingivalis, is an opportunistic oral pathogen most commonly associated with inception and progression of periodontal disease such as periodontitis, which is gingival inflammation with loss of connective tissues around teeth caused by undisturbed dental biofilm. Bacterial biofilms are a major cause of bacterial infections in humans, and remain one of the most significant challenges to human dental health.

Unfortunately, conventional therapies have shown to be inadequate in the treatment of most chronic biofilm infections based on the extraordinary innate tolerance of biofilms to antibiotics. Antagonists of quorum sensing (QS) signaling molecules have been used as means to control biofilms. QS and other cell-cell communication molecules are able to revert biofilm tolerance, prevent biofilm formation and disrupt fully developed biofilms, albeit with restricted effectiveness.

Recently, it has been found that hydrophobic active agents such as fatty acid signaling molecules play a vital role in the dispersion of biofilms formed by bacteria. For example, bacterium Pseudomonas aeruginosa produces a fatty acid messenger molecule cis-2-decenoic acid (cis-DA) that shows significant promise as an effective adjunctive to antimicrobial treatment of biofilms. Cis-DA is responsible for induction of the native biofilm dispersion response in a range of Gram-negative and Gram-positive bacteria and in yeast, and has been shown to reverse persistence, increase microbial metabolic activity and significantly enhance the cidal effects of conventional antimicrobial agents. It has been also reported that the use of cis-DA as a dispersing agent for biofilm control provided a solution to treating oral infections controlling biofilm formation (Marques, C.N.H., Pharmaceuticals, 2015, 8, 816-835). In particular, cis-DA has been shown to have cross-species and cross-kingdom biofilm dispersion activity in the oral cavity. In addition to its broad ability to induce dispersion, cis-DA has also been shown to prevent or limit attachment of cells and stunt formation of biofilms as well as awaken metabolically inactive, persister cells within biofilms. The dispersion and anti-biofilm activity of cis-DA against multiple different biofilm forming species has made it a target for development as an alternative therapeutic to traditional antibiotics for treating oral infections.

Despite the potential therapeutic function of hydrophobic active agents and/or fatty acid signaling molecules, there are practical challenges associated with the use of hydrophobic active agents. One significant barrier is the hydrophobicity of the agents, which unfavorably causes inadequate and/or inefficient delivery of the agents to oral cavity or target cells. It is thus highly desirable to improve the efficiency of delivering hydrophobic active agents and enhance the overall efficacy of treatment in preventing and dispersing biofilms. It is against the above background the present disclosure is provided.

### Enhanced Delivery of a Hydrophobic Agent in the Oral Cavity by Coupling to a Hydrophilic Agent with Cellular Activity

The present disclosure is related to compositions and methods for treating bacterial disease of the oral cavity. The composition can be formulated into delivery systems, including but not limited to: toothpaste, mouth-rinse, mouthwash, dentifrice and similar delivery devices. The composition generally includes a hydrophobic active agent selected from the group consisting of a bacterial biofilm dispersion agent, a bacterial biofilm inhibitor, and combinations thereof; and a hydrophilic agent. The hydrophilic agent may be a glycosaminoglycan. The composition provides the advantage of delivering a hydrophobic active agent to a target site with improved treatment efficacy.

The composition can include a paste, gel, powder, liquid dentifrice, prophylaxis paste, dental varnish, lozenge, mint, breath strip, chewing gum, chewable product, edible product, or other abrasive, detergent, or cleansing composition capable, either alone or in combination with the action of an applicator, of removing deposits, such as bacterial colonies from teeth and/or oral soft tissues.

It was surprisingly found that the efficacy of a hydrophobic active agent in preventing and dispersing biofilms is in large part dependent on successful delivery of the hydrophobic active agent to the target cells. In particular, the addition of a hydrophilic agent or carrier molecule to a treatment composition comprising a hydrophobic active agent can unexpectedly aid in delivery and targeting of the hydrophobic active agent to bacterial cells depositing on host tissues in the oral environment, thereby significantly improving the efficacy of the treatment composition. A particular but non-limiting example of the hydrophilic agent or carrier molecule is hyaluronic acid (HA). For example, an oral treatment composition comprising cis-DA and HA was found to have significantly improved efficacy compared with a composition not having both.

The oral treatment composition including a hydrophobic active agent and a hydrophilic agent can further include an antimicrobial agent that functions synergistically and compatibly with other components to improve the overall efficacy of the oral treatment composition.

In some aspects, the present disclosure relates to a method of treating an oral disorder comprising: delivering an oral treatment composition to a surface within the oral cavity, wherein the oral treatment composition comprises a hydrophobic active agent and a hydrophilic agent. In some embodiments, a method of treating an oral disorder comprises obtaining an oral treatment composition containing a hydrophobic active agent selected from the group consisting of a bacterial biofilm dispersion agent, a bacterial biofilm inhibitor, a fatty acid signaling agent, and combinations thereof; and a hydrophilic agent; and treating a surface within the oral cavity with the oral treatment composition. In preferred embodiments, the hydrophilic agent comprises a glycosaminoglycan. In embodiments, the hydrophobic agent is of about 0.00000001 to about 5 wt %, or from about 0.000001 wt % to about 2 wt %, or from about 0.00001 wt % to about 1 wt %, based on the total weight of the composition. In embodiments, the hydrophilic agent has a concentration from about 0.02 wt % to about 30 wt %, or from about 0.1 wt % to about 30 wt %, or from about 1 wt % to about 5 wt %, based on the total weight of the composition. In some embodiments, the composition of the present method further comprises an antimicrobial agent.

In some aspects, the present disclosure relates to a composition for treating an oral disorder comprising: a hydrophobic active agent and a hydrophilic agent. In some embodiments, a composition for treating an oral disorder comprises a hydrophobic active agent selected from the group consisting of a bacterial biofilm dispersion agent, a bacterial biofilm inhibitor, a fatty acid signaling agent, and combinations thereof; and a hydrophilic agent, thereby forming an oral treatment composition. In preferred embodiments, the hydrophilic agent comprises a glycosaminoglycan. In embodiments, the hydrophobic agent is of about 0.00000001 to about 5 wt %, or from about 0.000001 wt % to about 2 wt %, or from about 0.00001 wt % to about 1 wt %, based on the total weight of the composition. In embodiments, the hydrophilic agent has a concentration from about 0.02 wt % to about 30 wt %, or from about 0.1 wt % to about 30 wt %, or from about 1 wt % to about 5 wt %, based on the total weight of the composition. In some embodiments, the present composition further comprises an antimicrobial agent. In embodiments, the composition consists essentially of a hydrophobic active agent, a hydrophilic agent, and a carrier. Non-limiting examples of the carrier are water, aqueous solution, buffer, solvent, or combinations thereof.

### Definitions

As used herein, “weight percent,” “wt %, “percent by weight,” “% by weight,” and variations thereof refer to the concentration of a substance as the weight of that substance divided by the total weight of the composition and multiplied by 100. It is understood that, as used here, “percent,” “%,” and the like are intended to be synonymous with “weight percent,” “wt %, etc.

As used herein, “g” represents gram; “L” represents liter; “mg” represents “milligram (10⁻³ gram);” “mL” represents milliliter (10⁻³ liter); “nm” represents nanometer (10⁻⁹ meter); micrometer is 10⁻⁶ meter. The units “mg/100 g,” “mg/100 mL,” or “mg/L” are units of concentration or content of a component in a composition. One “mg/L” equals to one ppm (part per million). “Da” refers to Dalton, which is the unit for molecular weight; One Da equals to one g/mol. The unit of temperature used herein is degree Celsius (° C.).

The term “about” is used in conjunction with numeric values to include normal variations in measurements as expected by persons skilled in the art, and is understood to have the same meaning as “approximately” and to cover a typical margin of error, such as +10% of the stated value. The term “about” also encompasses amounts that differ due to different equilibrium conditions for a composition resulting from a particular initial composition. Whether or not modified by the term “about,” the claims include equivalents to the quantities.

It should be noted that, as used in this specification and the appended claims, the singular forms “a,” “an,” and “the” include plural referents unless the content clearly dictates otherwise. Thus, for example, reference to a composition containing “a compound” includes having two or more compounds that are either the same or different from each other. It should also be noted that the term “or” is generally employed in its sense including “and/or” unless the content clearly dictates otherwise.

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

The term “substantially free” may refer to any component that the composition of the disclosure lacks or mostly lacks. When referring to “substantially free” it is intended that the component is not intentionally added to compositions of the disclosure. Use of the term “substantially free” of a component allows for trace amounts of that component to be included in compositions of the disclosure because they are present in another component. However, it is recognized that only trace or de minimus amounts of a component will be allowed when the composition is said to be “substantially free” of that component. Moreover, the term if a composition is said to be “substantially free” of a component, if the component is present in trace or de minimus amounts it is understood that it will not affect the effectiveness of the composition. It is understood that if an ingredient is not expressly included herein or its possible inclusion is not stated herein, the disclosure composition may be substantially free of that ingredient. Likewise, the express inclusion of an ingredient allows for its express exclusion thereby allowing a composition to be substantially free of that expressly stated ingredient.

The methods, systems, and compositions of the present disclosure may comprise, consist essentially of, or consist of the components and ingredients of the present invention as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions. The term “hyaluronic acid” or “hyaluronan” as used herein generally includes a (co)polymer of acetylglucosamine (C₈H₁₅NO₆) and glucuronic acid (C₆H₁₀O₇) occurring as alternating units. Hyaluronic acid used herein encompasses any salts or derivatives thereof. Hyaluronic acid can be a natural product or a synthetic product or synthetic modification of a natural product.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosure. In the following description, various embodiments of the present disclosure are described with reference to the following drawings, in which:

FIGS. 1A and 1B illustrate the efficacy of example treatments according to Table 1 and the impact of cis-DA and HA against mature P. aeruginosa biofilms. FIG. 1A shows the log reduction in remaining cells of each treatment compared to the media only control (comparative example 5); FIG. 1B shows the percent reduction in remaining cells of each treatment compared to the media only control (comparative example 5).

FIG. 2 illustrates the impacts of cis-DA and HA on attachment of P. aeruginosa according to example treatments of Table 2.

### DETAILED DESCRIPTION

The compounds herein may be used to form an oral treatment composition. An oral treatment composition according to the present disclosure may comprise a mixture of different ingredients that when combined together result in an improved delivery mechanism to treat teeth and soft tissues of the oral cavity. The composition and treatment generally comprise a hydrophobic active agent selected from the group consisting of a bacterial biofilm dispersion agent, a bacterial biofilm inhibitor, a fatty acid signaling agent, and combinations thereof; and a hydrophilic agent. In preferred embodiments, the hydrophilic agent comprises a glycosaminoglycan. Generally the present composition can be used to treat an oral disorder by delivering an oral treatment composition to a surface within the oral cavity. The oral treatment composition according to the present disclosure may comprise a mixture of different variations of the compounds described herein and may further comprise additional ingredients that would be apparent to a skilled artisan in view of the disclosure to further provide improved efficacy and delivery.

An oral treatment composition described herein are selected from a group consisting of, a dentifrice (e.g., paste, gel, powder, or liquid dentifrice), prophylaxis paste, dental varnish, lozenge, chewing gum, or other abrasive, detergent, or cleansing composition capable, either alone or in combination with the action of an applicator, of removing deposits, such as plaque, from teeth and/or from oral soft tissues. Similar solid, semi-solid, and liquid formats may be useful for other topical uses. In alternative embodiments, the compositions described herein can be formulated for systemic and topical delivery (tablets, capsules, ointments, creams, pre-gels, lavages, solutions and suspension).

Alternatively, a topical or oral composition may be designed as a treatment composition, such as a liquid-gel, slurry, or suspension mouthwash or mouth-rinse. In oral, topical, and systemic somatic uses, the composition will respectively comprise orally, topically, or systemically acceptable substances. Such substances include a solid, semi-solid, or liquid carrier, and may optionally include one or more: other active ingredients, e.g., antibacterial agents (e.g., a water-insoluble non-cationic antibacterial agent, such as triclosan), antioxidants, pharmaceuticals, vitamins, fluoride sources, nutraceuticals, and the like; excipients and inert ingredients, e.g., humectants, gelling agents, thickeners, solvents, diluents, binders, fillers, plasticizers, anti-caking agents, disintegrants, gums, emollients, oleochemicals, colorants, flavorants, odorants, pH adjusting agents (acids, bases), buffers, surfactants, emulsifiers, suspending agents, enzymes, coatings (e.g., enteric, acrylic, or carbohydrate or cellulosic coatings), chelants, preservatives, and the like. Representative examples of such well-known additives are described, e.g., in J. G. Hardman et al., Goodman & Gilman's The Pharmacological Basis of Therapeutics (10^{th} ed., 2001); and R. C. Rowe et al., Handbook of Pharmaceutical Excipients (4^{th} ed., 2003); all of which are hereby incorporated by reference in their entirety.

A composition may optionally comprise a carrier. Carriers are commonly water, aqueous humectant, and/or aqueous alcohol mixtures of a consistency appropriate for the selected mode of administration of the composition, e.g., as a paste, gel, tablet, lozenge, syrup, rinse, and so forth. Carriers for oral compositions according to the present invention include all known in the art. Such orally acceptable carriers include the usual components of toothpastes, tooth powders, prophylaxis pastes, mouth-rinses, lozenges, gums and the like, and are more fully described hereinafter. Selection of specific carrier components is dependent on the desired product form, including dentifrices, rinses, gels, and confectionaries. One of skill in the art should appreciate that the addition of a carrier would be in addition to the hydrophilic agent described herein. The secondary additional carrier would be included for stability or enhancement of the delivery of the hydrophobic agent.

In various embodiments, the composition comprising a hydrophobic active agent (i.e. a bacterial biofilm dispersion agent, a bacterial biofilm inhibitor and combinations thereof) and a hydrophilic agent, such as glycosaminoglycan, is combined with an additional carrier used to prepare an oral composition and treatment. As recognized by one of skill in the art, the oral compositions optionally include other materials, such as for example, viscosity modifiers, diluents, surface active agents, such as surfactants, emulsifiers, and foam modulators, pH modifying agents, abrasives, humectants, emollients, and moisturizers, mouth feel agents, sweetening agents, flavor agents, colorants, preservatives and combinations thereof. It is understood that while general attributes of each of the above categories of materials may differ, there may be some common attributes and any given material may serve multiple purposes within two or more of such categories of materials. Preferably, such additional carrier materials are selected for compatibility with the active agent so as to not substantially minimize or reduce efficacy or treatment performance.

In the case of mouthwashes, sprays, or rinses, orally acceptable typically comprise an aqueous phase comprising water or a water and alcohol mixture. Further, in various embodiments, the oral carrier comprises a humectant and/or a surfactant. Generally, the weight ratio of water to alcohol is in the range of from about 1:1 to about 20:1, preferably about 3:1 to 10:1 and more preferably about 4:1 to about 6:1. The total amount of water-alcohol mixture in this type of preparation is typically in the range of from about 70 to about 99.9% of the preparation. In various embodiments, the alcohol is typically ethanol or isopropanol. The pH of such liquid and other preparations of the invention is generally in the range of from about 4.5 to about 10, which can be achieved and/or maintained with a pH control agent (acid or base) and/or a buffer such as sodium citrate, benzoate, carbonate, or bicarbonate.

In one embodiment, a composition, as formulated for application in the selected end-use, according to the present application will comprise an amount of active ingredients that consist of about 35% or less by weight of the final oral treatment composition, or about or less than 15 wt %, 12 wt %, 10 wt %, or 5 wt % of the oral composition. In one embodiment, the alternative ingredients will comprise from about 1 wt % to about 99 wt % by weight of the final composition, more preferably about 70 wt % or more. Active compounds can be provided as pre-mixes, kits or separate single portions to prepare a final formulation for use. In other embodiments, the active ingredients can comprise more than 0.02 wt %, and up to about 35 wt % of the weight thereof.

Preferably, an orally acceptable treatment composition, comprising the active ingredients, is applied to the oral cavity by contacting oral surface(s) with the treatment composition. In an embodiment, active ingredients introduced into the oral cavity, either during or after cleansing of oral surfaces, can remain resident upon cleansed oral surfaces to inhibit or reduce bacterial attachment, plaque formation, and the like. Similarly, treatment compositions hereof may also be used to prevent or reduce biofilm formation or microbial adhesion. The oral treatment compositions can also be used as bactericidal enhancers, such as for use on re-usable components used in dental and healthcare procedures. In particular, for dental and healthcare procedures performed in the oral cavity.

In one example method, a treatment composition comprises a hydrophobic active agent and a hydrophilic agent, thereby forming the active ingredients of the oral treatment composition. In some exemplary embodiments, the hydrophobic agent includes a bacterial biofilm dispersion agent, a bacterial biofilm inhibitor and combinations thereof. In addition, the hydrophilic agent may comprise a glycosaminoglycan. Combining these two active ingredients will form the treatment composition. Once prepared, the treatment composition is applied to a surface, for treating the surface within the oral cavity with the oral treatment composition.

In related embodiments, specific oral treatment compositions can include specific ingredients where the hydrophobic active agent is a bacterial biofilm dispersion agent. In at least one example embodiment the bacterial biofilm dispersion agent is cis-2-decenoic acid (cis-DA), recombinant DNase, alpha-amylase, Dsp B, D-amino acid, tetradecanoic acid, palmic acid, 9,12-linoleic acid, 9-oleic acid, 10-oleic acid, octadecoic acid, 7,10-oleic acid, 5,8,11,14-arachadonic acid, 7,10,13-eicosatrienoic acid, SPRE, or nitric oxide.

In alternative embodiments, the hydrophobic active agent comprises a bacterial biofilm inhibitor. In at least one example embodiment the bacterial biofilm inhibitor is a N-acyl homoserine lactone, an AI-2 inhibitor, a PQS inhibitor, nitric oxide, an imidazole derivative, an indole derivative, a 4-thiazolidinone pyrrole derivative or carolacton. In other related embodiments, the bacterial biofilm inhibitor is a statin. In at least one example embodiment the statin compounds that may be used are selected from a group comprising atorvastatin, simvastatin, lovastatin, fluvastatin, cerivastatin, pitavastatin and combinations thereof. In other related embodiments, a single statin. In another related embodiment, the single stain is used in combination with a hydrophilic agent.

In other embodiments, the hydrophobic active agent comprises one or more hydrophobic signaling agent. Non-limiting examples of hydrophobic signaling agent are fatty acid ligand, signaling lipids, hormones, hydrophobic signaling proteins. In particular, small chain fatty acid signaling ligands are important messengers for intracellular signal transduction. For example, cis-2-decenoic acid is able to change the status of many bacteria such as Pseudomonas aeruginosa and Escherichia coli persister cells from a dormant to a metabolically active state without an increase in cell number. Other examples of small chain fatty acids generally include any of C4-C22 fatty acids and derivatives or close modifications thereof. The fatty acids can have branched or unbranched structure, saturated or unsaturated C-C groups.

It should be appreciated that the use of statins provide a unique selective inhibition to certain bacteria from forming. Bacterial strains include, but are not limited to Pseudomonas, Porphyromonas aeruginosa, Porphyromonas gingivalis, Fusobacterium nucleatum, Tannerella forsythia, Actinomyces, Actinomyces naeslundii, Bacillus, Mycobacteria, Sphingomonas, Staphylococcus, and Streptococcus. and Streptococcus gordonii. In one example embodiment, at least one statin is used to selectively inhibit bacterial formation in an oral cavity. In a related embodiment, the specific bacterial strain inhibited is selected from a group consisting of Porphyromonas aeruginosa, Porphyromonas gingivalis, Fusobacterium nucleatum, Tannerella forsythia, Actinomyces naeslundii, and Streptococcus gordonii. In one example, embodiment the composition comprises simvastatin for selectively reducing Porphyromonas gingivalis in an oral cavity.

In some embodiments, the hydrophilic agent is a negatively charged polysaccharide, such as, for example, an animal- or plant-derived polysaccharide or a synthetically modified polysaccharide. As a non-limiting example of a plant-derived polymer, the negatively charged polysaccharide comprises a glycosaminoglycan, such as, for example, hyaluronic acid, chondroitin sulfate, or other acidic polymers such as dextran sulfate. In certain embodiments the hydrophilic agent is a glycosaminoglycan. In one examples embodiment the glycosaminoglycan is hyaluronic acid and hyaluronic acid derivatives. Hyaluronic acid is hydrophilic and is attracted to sites of inflammation. It can also deposit onto and into soft tissues. Further, hyaluronic acid also has some capacity to carry or cage smaller molecules due to its relatively large molecular size and configuration. In other embodiments, the glycosaminoglycan is chondroitin sulfate, dermatin sulfate, heparin, heparin sulfate, keratin sulfate, or combinations thereof.

In some embodiments, the glycosaminoglycan has an average molecular weight from about 4 kDa to about 8,000 kDa, or from about 4 kDa to about 4,000 kDa, or from about 4 kDa to about 2,000 kDa, or from about 4 kDa to about 1,000 kDa, or from about 4 kDa to about 800 kDa, or from about 4 kDa to about 400 kDa, or from about 4 kDa to about 200 kDa, or from about 4 kDa to about 100 kDa, or from about 4 kDa to about 50 kDa, or from about 10 kDa to about 8,000 kDa, or from about 50 kDa to about 8,000 kDa, or from about 100 kDa to about 8,000 kDa, or from about 200 kDa to about 8,000 kDa, or from about 500 kDa to about 8,000 kDa, or from about 1,000 kDa to about 8,000 kDa, or from about 2,000 kDa to about 8,000 kDa, or from about 4,000 kDa to about 8,000 kDa, or from about 6,000 kDa to about 8,000 kDa, or from about 100 kDa to about 2,000 kDa, or from about 500 kDa to about 1,000 kDa.

While not wishing to be bound to a particular theory, it is believed that the hydrophilic agent such as glycosaminoglycan or hyaluronic acid when combined with the hydrophobic active agent provides a unique mechanism to improve the delivery efficiency of the hydrophobic active agent to the target place and synergistically enhance the overall efficacy of the treatment.

In some example embodiments, an antimicrobial agent (e.g., biocide, antibiotic) is combined with the hydrophobic active agent and the hydrophilic agent. Examples include cephalosporins, penicillins, aminoglycosides, tetracyclines, clindamycin, chloramphenicol, macrolides, fluoroquinolones, vancomycin, actinomycin, metronidazole, lactic acid, sorbic acid, fluconazole, nystatin, chlorhexidine, benzalkonium chloride, benzoyl peroxide, hydrogen peroxide, hexachlorophene, phenol, resorcinol and cetylpyridinium chloride.

As mentioned above the final oral treatment composition can be formulated in a manner such as a mouthwash, toothpaste, and/or dentifrice. Once the oral treatment composition is prepared, it is applied to a surface, such as a diseased surface within the oral cavity. In other related embodiments the oral treatment composition is delivered to at least one periodontal pocket. In other related embodiments, the oral treatment composition is applied to a mouth arch to allow for extended exposure to surface in the oral cavity around the teeth and gingiva tissue.

Although not wanting to be bound by any particular theory the preferred concentrations of the active ingredients in an oral treatment composition can be determined by routine analysis of a series of test compositions containing, e.g. different concentrations of active ingredients combined with alternative ingredients, or not, under a series of conditions, such as the time of treatment. The rate or degree of inhibition of bacterial attachment to a mouth, or to an oral cavity model (such as an “artificial mouth”), treated with the compositions can be assessed by quantitative or semi-quantitative analysis, such as by a calorimetric technique to assess bacterial colony inhibition or reduction.

For example the hydrophobic agent could be a bacterial biofilm dispersion agent (e.g., cis-2-decenoic acid) or a bacterial biofilm inhibitor with a concentration of about 0.00000001 wt % to about 5 wt %, or from 0.000001 wt % to about 5 wt %, or from about 0.00001 wt % to about 5 wt %, or from about 0.0001 wt % to about 5 wt %, or from about 0.001 wt % to about 5 wt %, or from about 0.01 wt % to about 5 wt %, or from 0.1 wt % to about 5 wt %, or from about 0.00000001 wt % to about 4 wt %, or from about 0.00000001 wt % to about 3 wt %, or from about 0.00000001 wt % to about 2 wt %, or from about 0.00000001 wt % to about 1 wt %, or from about 0.00000001 wt % to about 0.1 wt %, or from about 0.00000001 wt % to about 0.01 wt %, or from about 0.00000001 wt % to about 0.001 wt %, or from about 0.00000001 wt % to about 0.0001 wt %, or from about 0.00000001 wt % to about 0.00001 wt %, or from about 0.00000001 wt % to about 0.000001 wt %, or from about 0.00000001 wt % to about 0.0000001 wt %, or from about 0.0000001 wt % to about 1 wt %, or from about 0.000001 wt % to about 1 wt %, or from about 0.00001 wt % to about 1 wt %, or from about 0.0001 wt % to about 1 wt %, or from about 0.001 wt % to about 1 wt %, or from about 0.01 wt % to about 1 wt %, or from 0.1 wt % to about 1 wt %, or from about 0.000001 wt % to about 2 wt %, or from about 0.0001 wt % to about 1 wt %, based on the total weight of the composition.

In addition the hydrophilic agent could be a glycosaminoglycan (e.g., hyaluronic acid, chondroitin sulfate, etc.), with a concentration range of about 0.02 wt % to about 30 wt %, or from about 0.02 wt % to about 20 wt %, or from about 0.02 wt % to about 10 wt %, or from about 0.02 wt % to about 5 wt %, or from about 0.02 wt % to about 3 wt %, or from about 0.02 wt % to about 1 wt %, or from about 0.02 wt % to about 1 wt %, or from about 0.02 wt % to about 0.1 wt %, or from about 0.05 wt % to about 30 wt %, or from about 0.1 wt % to about 30 wt %, or from about 1 wt % to about 30 wt %, or from about 5 wt % to about 30 wt %, or from about 10 wt % to about 30 wt %, or from about 20 wt % to about 30 wt %, or from about 0.1 wt % to about 10 wt %, or from about 1 wt % to about 5 wt %, based on the total weight of the composition.

### EXAMPLES

Certain embodiments of the present disclosure are further described with reference to the following examples. These examples are intended to be merely illustrative of the disclosure and are not intended to limit or restrict the scope of the present disclosure in any way and should not be construed as providing conditions, parameters, reagents, or starting materials that must be utilized exclusively in order to practice the art of the present disclosure.

Oral treatment experiments using compositions comprising HA and cis-DA were performed, and the impact of HA on efficacy of dispersion and biofilm formation was tested in comparison to treatment using cis-DA alone. The effect of HA on efficacy of cis-DA was tested against P. aeruginosa. It was noted that P. aeruginosa is native producer of cis-DA and the strain typically observes strong dispersion activity in response to cis-DA. These experiments revealed both the effect of HA and cis-DA on surface attachment and biofilm formation, and the effect of HA and cis-DA on dispersion of pre-established biofilms.

### Experiments and Methods

Bacterial Strains and Conditions

The bacterial strain used in this study was Pseudomonas aeruginosa PAO1. Primary cultures of P. aeruginosa were grown in full-strength Luria-Bertani (LB) broth. The cultures were inoculated from frozen stocks and placed in a shaking incubator at 37° C. and 220 rpm overnight (18 hr) before use in dispersion and attachment assays.

Semi-Batch Biofilm Growth

P. aeruginosa biofilms were grown using semi-batch biofilm growth techniques in 24-well plates. Plates were inoculated with overnight P. aeruginosa cultures adjusted to an OD₆₀₀ of 0.1 in 1:5 LB medium and placed in a shaking incubator at 37° C. and 100 rpm. After 24 hours, the supernatant was removed and replaced with fresh 1:5 LB. The plates were returned to their original growth conditions. Following the first 24 hr of growth and attachment, media was changed, as described above, twice a day for 2 additional days. Following 3 days of grown in this semi-batch system, mature biofilms were developed and ready for testing dispersion induction.

Dispersion Assays

Following mature biofilm development in the semi-batch system biofilms were tested for their dispersion efficacy in the presence and absence of cis-DA, HA, and cis-DA in combination with HA to observe the effect of HA in improving dispersion efficacy of cis-DA alone. For P. aeruginosa, on the third day of growth, the media was changed three hours before treatment to prevent nutrient-triggered dispersion from interfering with data collection. After three hours of incubation the supernatant from each of the wells was removed and replaced with treatment media. Six example treatments at a time were used according to Table 1. Once treatments were added, the plates were placed under original growing conditions for one hour. After one hour, the supernatant liquid containing dispersed cells was collected and remaining biofilms were re-suspended in 0.85% saline for viability counting. Re-suspended biofilms were homogenized to break up and homogenize biofilm cells for viability counting. Supernatant and biofilm solutions were serially diluted and then dropped onto 1:2 LB agar plates. The plates were incubated at 37° C. overnight and CFUs were counted to determine CFU/mL in the supernatant liquid and the remaining biofilm for each treatment. Amount of released or dispersed cells and remaining biofilms were expressed in log CFU/mL values, log increase in released cells, and percent increase in released cells as three, complementary representations of the data.

TABLE 1
Comparative examples 1-4 and examples 1-2 of
oral treatments against P. aeruginosa.

| | |
|---|---|
| Comparative example 1 | 1:5 LB only |
| (Media control) | |
| Comparative example 2 | 1:5 LB with cis-DA (310 nM) |
| (cis-DA control) | |
| Comparative example 3 | 1:5 LB with 0.5 wt % HA (832 kDa) |
| (HA control) | |
| Comparative example 4 | 1:5 LB with 0.2 wt % HA (832 kDa) |
| (HA control) | |
| Example 1 | 1:5 LB with cis-DA (310 nM) |
| | and 0.5 wt % HA |
| Example 2 | 1:5 LB with cis-DA (310 nM) |
| | and 0.2 wt % HA |

### Attachment Assays

To test the effects of cis-DA and HA on the prevention of biofilm growth, attachment assays were performed. Overnight cultures for P. aeruginosa attachment assays were grown in LB. 96-well plates were inoculated with primary cultures of P. aeruginosa that were adjusted to a final OD₆₀₀ of 0.02 in 1:20 LB medium. Six treatments were used according to Table 3. 1:20 LB only, 1:20 LB with cis-DA (310 nM), 1:20 LB with 0.2% HA, 1:20 LB with cis-DA (310 nM) and 0.2% HA, 1:20 LB with 0.5% HA, and 1:20 LB with cis-DA and 0.5% HA. Adjusted cultures with respective treatment media were then added to each well and the plates were placed in a shaking incubator at 37° C. and 220 rpm for 24 hr to assess irreversible attachment. Plates were allowed to dry overnight. Once dry, plates were observed under 200× magnification with a minimum of 4 wells and 10 fields of view captured for each treatment. Representative images were selected to show cell attachment for each strain.

TABLE 2
Comparative examples 5-8 and examples 3-4 of
oral treatments against P. aeruginosa.

| | |
|---|---|
| Comparative example 5 | 1:20 LB only |
| (Media control) | |
| Comparative example 6 | 1:20 LB with cis-DA (310 nM) |
| (cis-DA control) | |
| Comparative example 7 | 1:20 LB with 0.5 wt % HA (832 kDa) |
| (HA control) | |
| Comparative example 8 | 1:20 LB with 0.2 wt % HA (832 kDa) |
| (HA control) | |
| Example 3 | 1:20 LB with cis-DA (310 nM) |
| | and 0.5 wt % HA |
| Example 4 | 1:20 LB with cis-DA (310 nM) |
| | and 0.2 wt % HA |

### Results

### Impact of HA on Cis-DA Dispersion Efficacy

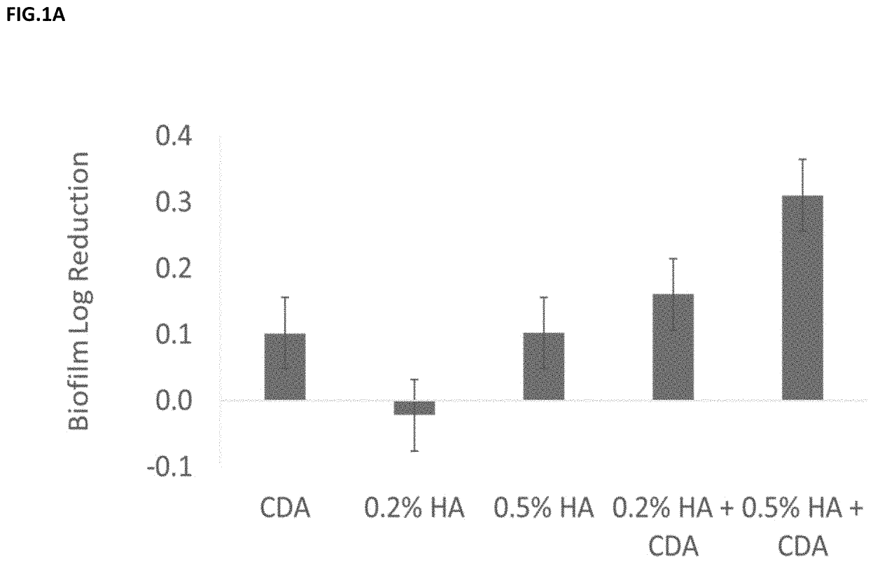

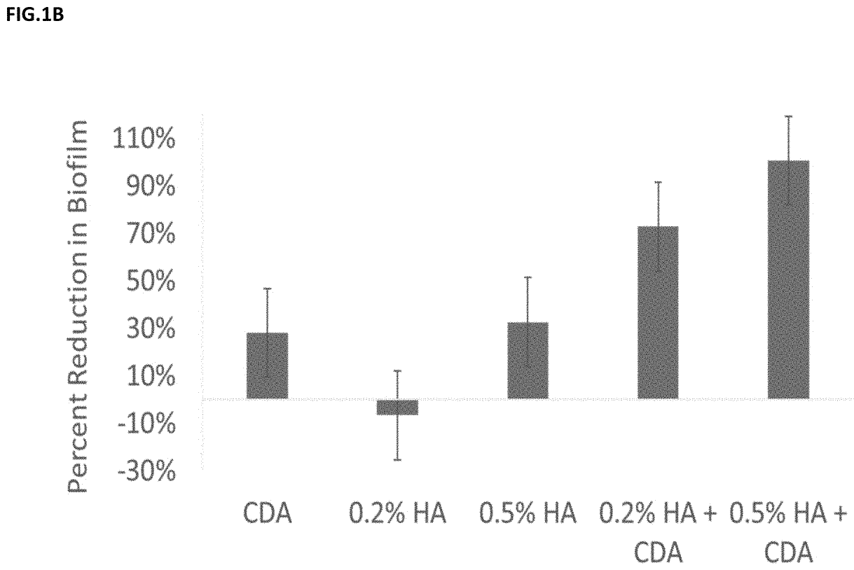

Referring to FIGS. 1A. 1B, and Table 1, P. aeruginosa biofilms were grown in a semi-batch system in 24-well plates for a period of 3 days to allow development of mature biofilms. Biofilms were dispersed by replacing medium with various media containing cis-DA, hyaluronic acid, and a combination of cis-DA and hyaluronic acid. Treatments were compared to media controls. Treatments were exposed to biofilms for a period of 1 hour under standard growth conditions. Following treatment, the supernatant liquid was removed and the remaining biofilm was collected and measured using viable plate counting. Data represent 7 independent trials. FIG. 1A shows the log reduction in remaining cells of each treatment compared to the media only control (comparative example 1); FIG. 1B shows the percent reduction in remaining cells of each treatment compared to the media only control (comparative example 1).

When looking at the remaining biofilms following dispersion treatment, we see positive impact for treatments with both cis-DA and HA (Examples 1 and 2) in reducing the remaining biofilm biomass (FIG. 1). As can been seen from FIG. 1A (log reduction) or FIG. 1B (percent decrease), the effects of HA on cis-DA's ability to decrease biofilm biomass are considerable. These data indicate that the combinations of cis-DA and HA may reduce the overall biomass of the remaining mature biofilm.

Impact of HA on Cis-DA Anti-Biofilm Activity

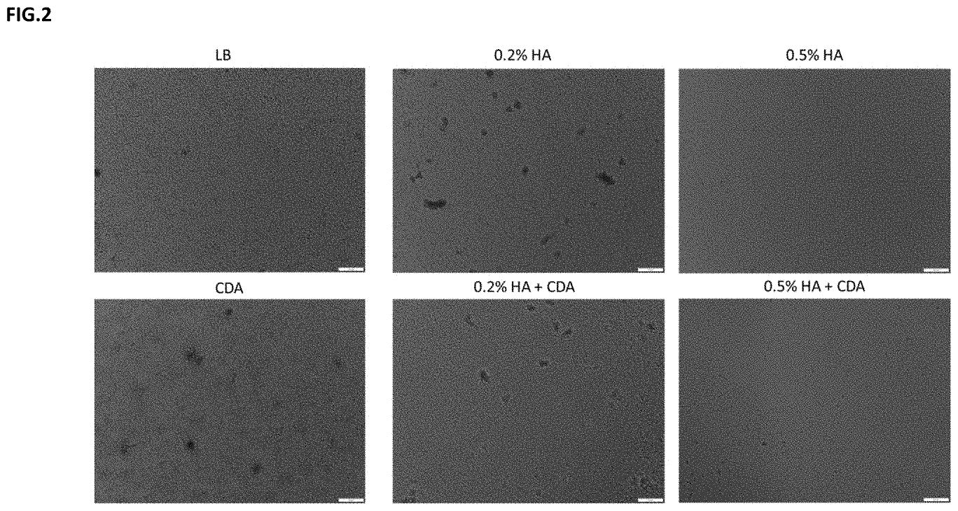

Referring now to FIG. 2 and Table 2, where P. aeruginosa cells were allowed to attach to 96-well plates for a period of 24 hr in growth medium alone or growth medium containing cis-DA, hyaluronic acid, and a combination of cis-DA and hyaluronic acid. Plates were allowed to dry and attached cells were observed via microscopy under 200× magnification. Data shown are representative images of the number of attached cells following 24 hr of growth in a particular treatment. In P. aeruginosa experiments, when visualizing the attached cells using microscopy, a thinner biofilm monolayer and fewer attached cells were observed after treatment with cis-DA and HA (FIG. 2).

The above specification, examples and data provide a complete description of the manufacture and use of the composition of the invention. Since many embodiments of the invention can be made without departing from the spirit and scope of the invention, the invention resides in the claims hereinafter appended.

1. A method of treating an oral disorder comprising: delivering an oral treatment composition to a surface within an oral cavity, wherein the oral treatment composition comprises a hydrophobic active agent and a hydrophilic agent.

2. (canceled)

3. The method of claim 1, wherein the hydrophobic active agent is a bacterial biofilm dispersion agent.

4. The method of claim 3, wherein the bacterial biofilm dispersion agent is selected from a group consisting of cis-2-decenoic acid, recombinant DNase, alpha-amylase, Dsp B, D-amino acid, tetradecanoic acid, palmic acid, 9,12-linoleic acid, 9-oleic acid, 10-oleic acid, octadecoic acid, 7,10-oleic acid, 5,8,11,14-arachadonic acid, 7,10,13-eicosatrienoic acid, SPRE, nitric oxide, and combinations thereof.

5. The method of claim 1, wherein the hydrophobic active agent is a bacterial biofilm inhibitor.

6. The method of claim 5, wherein bacterial biofilm inhibitor is selected from a group consisting of a N-acyl homoserine lactone, an AI-2 inhibitor, a PQS inhibitor, nitric oxide, an imidazole derivative, an indole derivative, a 4-thiazolidinone pyrrole derivative, carolacton, atorvastatin, simvastatin, lovastatin, fluvastatin, cerivastatin, pitavastatin and combinations thereof.

7. The method of claim 1, wherein the hydrophobic agent comprises one or more fatty acid signaling molecule having from about 4 to about 22 carbons.

8. The method of claim 1, wherein the hydrophilic agent is a glycosaminoglycan.

9. The method of claim 8, wherein the glycosaminoglycan is hyaluronic acid or a derivative thereof.

10. (canceled)

11. (canceled)

12. (canceled)

13. (canceled)

14. (canceled)

15. (canceled)

16. (canceled)

17. The method of claim 1, wherein the hydrophobic agent has a concentration of about 0.00000001 wt % to about 5 wt %, or from about 0.000001 wt % to about 2 wt %, or from about 0.00001 wt % to about 1 wt %.

18. The method of claim 1, wherein the hydrophilic agent has a concentration range of about 0.02 wt % to about 30 wt %, or from about 0.1 wt % to about 10 wt %, or from about 1 wt % to about 5 wt %.

19. The method of claim 1, wherein the composition further comprises an antimicrobial agent selected from a group consisting of cephalosporins, penicillins, aminoglycosides, tetracyclines, clindamycin, chloramphenicol, macrolides, fluoroquinolones, vancomycin, actinomycin, metronidazole, lactic acid, sorbic acid, fluconazole, nystatin, chlorhexidine, benzalkonium chloride, benzoyl peroxide, hydrogen peroxide, hexachlorophene, phenol, resorcinol, cetylpyridinium chloride and combinations thereof.

20. A composition for treating an oral disorder comprising:
a hydrophobic active agent; and
a hydrophilic agent.

21. (canceled)

22. (canceled)

23. The composition of claim 20, wherein the hydrophobic active agent is a bacterial biofilm dispersion agent.

24. The composition of claim 22, wherein the bacterial biofilm dispersion agent is selected from a group consisting of cis-2-decenoic acid, recombinant DNase, alpha-amylase, Dsp B, D-amino acid, tetradecanoic acid, palmic acid, 9,12-linoleic acid, 9-oleic acid, 10-oleic acid, octadecoic acid, 7,10-oleic acid, 5,8,11,14-arachadonic acid, 7,10,13-eicosatrienoic acid, SPRE, nitric oxide and combinations thereof.

25. The composition of claim 20, wherein the hydrophobic active agent is a bacterial biofilm inhibitor.

26. The composition of claim 25, wherein bacterial biofilm inhibitor is selected from a group consisting of a N-acyl homoserine lactone, an AI-2 inhibitor, a PQS inhibitor, nitric oxide, an imidazole derivative, an indole derivative, a 4-thiazolidinone pyrrole derivative, carolacton, atorvastatin, simvastatin, lovastatin, fluvastatin, cerivastatin, pitavastatin and combinations thereof.

27. The composition of claim 20, wherein the hydrophobic agent comprises one or more fatty acid signaling molecule having from about 4 to about 22 carbons.

28. The composition of claim 20, wherein the hydrophilic agent is a glycosaminoglycan.

29. The composition of claim 28, wherein the glycosaminoglycan is hyaluronic acid or a derivative of hyaluronic acid.

30. (canceled)

31. (canceled)

32. (canceled)

33. (canceled)

34. (canceled)

35. (canceled)

36. The composition of claim 20, wherein the oral treatment composition further comprises an antimicrobial agent.

37. (canceled)

38. (canceled)

39. (canceled)
